(19) United States
(12) Patent Application Publication
(10) Pub. No.: US 2021/0293698 A1
(43) Pub. Date: Sep. 23, 2021

(54) SYNTHESIS OF GOLD NANOPARTICLES FUNCTIONALIZED WITH HYDROXYPYRIDINONE CHELATORS FOR METAL SENSING

(71) Applicant: THE REGENTS OF THE UNIVERSITY OF CALIFORNIA, Oakland, CA (US)

(72) Inventors: Roger M. Pallares, Berkeley, CA (US); Rebecca J. Abergel, Kensington, CA (US)

(73) Assignee: THE REGENTS OF THE UNIVERSITY OF CALIFORNIA, Oakland, CA (US)

(21) Appl. No.: 17/154,317

(22) Filed: Jan. 21, 2021

Related U.S. Application Data
(60) Provisional application No. 62/964,789, filed on Jan. 23, 2020.

(51) Int. Cl. G01N 21/33

(52) U.S. Cl. CPC G01N 21/33

(57) ABSTRACT

A colorimetric assay of lanthanide or actinide (f-block elements) samples and a sensing construct of functionalized gold nanoparticles are provided. The construct is a gold nanoparticle with hydroxypyridinone ligands on the surface. The colorimetric assay can be performed in two different ways: (1) Gold nanoparticles functionalized with hydroxypyridinone ligands are synthesized, and then added to the sample solution. The presence of f-block elements triggers the gold nanoparticle aggregation, which changes the solution color. (2) Exploiting the competition between Au³⁺ and cations with higher binding affinities, where the f-block elements quench the nanoparticle growth and affect the color of solution. In both methods, the change in color is used as a sensing principle to quantify the f-block element concentrations in aqueous solutions.

### CROSS-REFERENCE TO RELATED APPLICATIONS

This application claims priority to, and the benefit of, U.S. provisional patent application Ser. No. 62/964,789 filed on Jan. 23, 2020, incorporated herein by reference in its entirety.

### STATEMENT REGARDING FEDERALLY SPONSORED RESEARCH OR DEVELOPMENT

This invention was made with government support under Contract No. DE-AC02-05CH11231 awarded by the U.S. Department of Energy. The government has certain rights in the invention.

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND

### 1. Technical Field

This technology pertains generally to chemical sensing and more particularly to systems and methods for lanthanide and actinide (i.e., f-block elements) sensing and quantification with metal nanoparticles functionalized with hydroxypyridinone ligands with the octadentate hydroxypyridinone chelator 3,4,3-LI(1,2-HOPO) particularly preferred.

### 2. Background

Modern technologies ranging from smartphones to wind turbines are critically dependent on rare earth elements (REE) that include the lanthanide series of elements, with atomic numbers beginning from 57 and ending at 71, and the actinide elements, with atomic numbers beginning from 89 and ending at 103. REE elements are important for technological innovation due to their unique magnetic, luminescence, and catalytic properties. For example, Gd is used as a contrasting agent in magnetic resonance imaging (MRI), Nd is used as magnets for disk drives and Ce is used as a catalyst.

The REE metals are being extracted and consumed at an unprecedented rate worldwide. These activities, along with inadequate disposal and recycling attempts, can create significant environmental contaminations of air, soil and water and potentially toxic exposures of people to significant quantities of REE's. Consequently, adequate monitoring and protection measures must be utilized to control human and environmental exposures to REE's. To date there have been some attempts to develop detection methods for various REE metal ions from a variety of samples from groundwater to human tissues. However, existing methods rely on instrumentation that is intensive in cost and time or rely on schemes that lack specificity and sensitivity.

Siderophores are a group of biomolecules produced by bacteria, fungi and some plants that transport iron across cell membranes. Bioinspired siderophore analogues that contain hydroxypyridinone (HOPO) groups, such as 3,4,3-LI(1,2-HOPO), are known for their high affinity for f-block cations. As 3,4,3-LI(1,2-HOPO) can be orally-administered and is non-cytotoxic at medical dosages, it is primarily developed as a chelating agent for the decorporation of radionuclides and as a treatment for heavy metal poisoning. In addition, the high stability and fast excretion kinetics of HOPO complexes with alpha-emitting isotopes have resulted in the pursuit of new radiopharmaceutical agents for cancer therapy, and HOPO moieties are also known to sensitize the luminescence of several lanthanides, which can be applied to bioassays. While the coordination chemistry of 3,4,3-LI(1,2-HOPO) with f-block cations has been thoroughly investigated, the complexation of noble metals, such as gold, and their applications have yet to be explored.

Gold has been used in medicine throughout history from dentistry to arthritis treatments. When the dimensions of metallic gold are reduced to nanoscale, such as in the form of gold nanoparticles (AuNPs), different size and shape-dependent physicochemical properties arise. Since AuNP optical properties are sensitive to the particle surroundings, they can be used as transducers for sensitive bioassays. Several chelating agents have been functionalized on the AuNP surface for the development of therapeutics and sensing agents, however, the functionalization of chelators on AuNPs usually requires tedious multi-step reactions. Simultaneous one-pot synthesis and functionalization of AuNPs with chelating agents is a preferred approach, yet this has only been successful with a limited suite of ligands including ethylenediaminetetraacetic acid (EDTA).

Accordingly, there is a need for increasingly sensitive and sophisticated methods for the detection and quantification of REE's that are non-toxic, reliable and have relatively few process steps.

### BRIEF SUMMARY

Systems and methods are provided for selective and sensitive lanthanide and actinide sensing using metal nanoparticles functionalized with chelators produced with a one-pot synthesis scheme. Biologically inert metal nanoparticles functionalized with chelators have demonstrated great potential in sensing and biomedicine, yet the chelator functionalization usually requires tedious multi-step reactions. Functionalized nanoparticles display different optical responses after binding to lanthanides and actinides and that optical response can be employed in their analysis.

Methods are provided that combine one-pot synthesis and functionalization of AuNPs with better performing chelators specific for f-block elements that are suited for the development of new nano-constructs for biomedical applications. For example, the octadentate hydroxypyridinone chelator 3,4,3-LI(1,2-HOPO) is a promising therapeutic agent because of its high affinity for f-block elements and non-cytotoxicity at medical dosages.

The methods are illustrated with high performing 3,4,3-LI(1,2-HOPO) and colloidal gold. However, other ligands including other hydroxypyridinone ligands such as TREN-(Me-3,2-HOPO) and 5-LIO(Me-3,2-HOPO) can be used.

The protocols synthesize monodisperse gold nanoparticles that display active 3,4,3-LI(1,2-HOPO) chelator molecules on the particle surface. The preferred protocol only requires one step: mixing equimolar amounts of 3,4,3-LI(1,2-HOPO) and chloroauric acid (HAuCl₄) and hydrogen chloride to adjust the pH between 2.4 and 5.9 (optimal pH 4.9) to obtain the gold nanoparticles.

The functionalized gold nanoparticles that are produced can be used as obtained or washed and re-dispersed in water. Because they display the 3,4,3-LI(1,2-HOPO) chelator, for example, they selectively bind to lanthanides and actinides, which cause a change in the nanoparticle optical properties that can be used for detection of these elements.

The thermodynamic properties of the solution of complexes formed between 3,4,3-LI(1,2-HOPO) and gold(III) ions have been characterized. It is shown that the chelator promotes the growth of gold nanoparticles under a specific range of pH conditions, acting as both reducing and stabilizing agent.

Au³⁺ complexation is observed even at acidic pH (2.2), where most of the HOPO binding units were protonated. Therefore, only a small fraction of partially deprotonated 3,4,3-LI(1,2-HOPO) is necessary to trigger complexation, indicative of high thermodynamic stability between the cation and the chelator. Above pH 2.4, complexation was followed by the reduction of Au³⁺ to Au⁰ and subsequent formation of AuNPs. During the growth of the particles, 3,4,3-LI(1,2-HOPO) acts as both reducing and stabilizing agent. The 3,4,3-LI(1,2-HOPO) ligands on the nanoparticle surface were observed to remain active and selective towards f-block elements, as evidenced by gold nanoparticle selective aggregation.

Finally, a new colorimetric assay is provided that is capable of reaching the detection levels necessary for the quantification of lanthanides (and actinides) in waste from industrial processes that is based on the inhibition of particle growth by lanthanides. The colorimetric assay can be performed in two different ways: (1) Gold nanoparticles functionalized with 3,4,3-LI(1,2-HOPO) are synthesized, and then added to the lanthanide or actinide solution. The presence of f-block elements triggers the gold nanoparticle aggregation, which changes the solution color. (2) Exploiting the competition between Au³⁺ and cations with higher binding affinities, where the f-block elements quench the nanoparticle growth and affect the color of solution. In both protocols, the change in color is used as a sensing principle to quantify the f-block element concentrations in aqueous solutions. The tunable dynamic range of the colorimetric assay covers a wide range of concentrations relevant to the detection of f-block elements in waste from industrial processes.

In one embodiment, the functionalized metal nanoparticle sensor is synthesized with a one-pot method comprising: mixing equimolar amounts of 3,4,3-LI(1,2-HOPO) and Chloroauric Acid (HAuCl₄); adjusting the pH to between approximately pH 2.4 and approximately pH 5.9; allowing the reaction to progress for a period of time and collecting the resulting monodisperse gold nanoparticles.

The functionalized nanoparticle sensors can be used for detecting the presence or absence of a lanthanide or actinide elements in a sample. The steps of the method comprise mixing the functionalized nanoparticle sensors with a sample solution of a lanthanide or actinide of unknown concentration; allowing the mixture to incubate and react undisturbed for a period of time to produce an incubated mixture and detecting the presence or absence of lanthanides or actinides and quantifying the concentration of detected lanthanides or actinides by either measuring solution absorbance by UV-Vis spectroscopy or by visual observance of solution color and then comparing the color to a reference of colors of known concentrations.

In another embodiment, the methods quantify the concentration of lanthanides or actinides in a sample solution by mixing equimolar amounts of 3,4,3-LI(1,2-HOPO) and Chloroauric Acid (HAuCl₄) to the solution sample containing lanthanides or actinides; adjusting the pH of the combined solution to between approximately pH 2.4 and approximately pH 5.9, with pH 4.9 optimal; and allowing the reaction to progress for a period of time. The presence and concentration of lanthanides or actinides can be quantified visually by comparing the color to a reference or by measuring the resulting solution absorbance by UV-Vis spectroscopy or other suitable absorbance detection technology.

Unless defined otherwise, all technical and scientific terms used herein have the same meaning as commonly understood by one of ordinary skill in the art to which this invention is related. The following terms are defined for purposes of the invention as described herein.

According to one aspect of the technology, a sensor construct is provided of gold nanoparticles functionalized with hydroxypyridinone chelators that can reliably detect and quantify f-group elements and is inexpensive to produce and use.

Another aspect of the technology is to provide a simple assay for detecting and quantifying lanthanide and actinides in a sample using gold nanoparticles functionalized with hydroxypyridinone chelators where the presence of f-block elements triggers the gold nanoparticle aggregation and a change in the color of the solution.

A further aspect of the technology is to provide a method that exploits the competition between Au³⁺ and cations with higher binding affinities, where the f-block elements quench the nanoparticle growth and affect the color of the solution as a sensing principle to quantify f-block element concentrations in aqueous solutions.

Another aspect of the technology is to provide a colorimetric assay with a tunable dynamic range that can quantify a wide range of concentrations relevant to the detection of lanthanide or other f-block elements in waste from industrial processes.

Further aspects of the technology described herein will be brought out in the following portions of the specification, wherein the detailed description is for the purpose of fully disclosing preferred embodiments of the technology without placing limitations thereon.

### BRIEF DESCRIPTION OF THE SEVERAL VIEWS OF THE DRAWINGS

The technology described herein will be more fully understood by reference to the following drawings which are for illustrative purposes only:

FIG. 1 is a schematic flow diagram of a method for synthesizing functionalized monodisperse gold nanoparticles that display active 3,4,3-LI(1,2-HOPO) chelator on the particle surface according to one embodiment of the technology.

FIG. 2 is a schematic flow diagram of a method for detecting and quantifying lanthanides or actinides using synthesized nanoparticles added into a sample containing the lanthanide. The higher the concentration of lanthanides, the stronger the nanoparticle color change becomes (red to blue) according to one embodiment of the technology.

FIG. 3 is a schematic flow diagram of a method for detecting and quantifying lanthanides or actinides using a single pot synthesis and detection method. The nanoparticles are grown in situ in the sample solution. Because the lanthanides or actinides quench the growth of gold nanoparticles, the higher the lanthanide concentration, the less intense the nanoparticle color becomes (from strong red to colorless) according to an alternative embodiment of the technology.

FIG. 4 is a structure diagram of a 3,4,3-LI(1,2-HOPO) chelator.

FIG. 5 is a graph depicting the spectrophotometric titration for the Au³⁺-3,4,3-LI(1,2-HOPO) complex. The concentration of ligand was 400 μM, and the concentration of Au³⁺ ranged from 0 to 400 μM. The pH of the solution was 2.2 with an ionic strength of 0.1 M (KCl).

FIG. 6 is UV-vis spectra of 1:1 mixtures of Au³⁺ and 3,4,3-LI(1,2-HOPO) at different pH values. The concentration of ligand and Au3+ was 400 μM in 100 μL of solution. The UV-vis spectra have been offset for clarity.

FIG. 7 is a UV-vis spectrum of AuNPs grown with 3,4,3-LI(1,2-HOPO) at pH 4.9 indicated the nanoparticles are primarily monodisperse in solution.

FIG. 8 is UV-Vis spectra of AuNPs with different cations (10 μM) demonstrating the preserved selectivity of the towards f-block elements of the AuNP functionalized with 3,4,3-LI(1,2-HOPO). The pH of the solutions was kept at 6.0 with MES buffer (0.1 M).

FIG. 9 depicts UV-vis spectra of particle growth kinetics of AuNPs at pH 4.9.

FIG. 10 is a graph of extinction variation at 526 nm during particle growth. The concentration of ligand and Au³⁺ was 400 μM in 1 mL of growth solution.

FIG. 11 is a graph of UV-vis spectra of AuNPs grown after addition of Eu³⁺ in competition experiments with Eu³⁺. The final volume of the solutions was 100 μL. The inset is the picture of AuNP solution grown in the presence of 0, 200, and 400 μM Eu³⁺.

FIG. 12 is a graph depicting variation of the extinction intensity at LSP band maxima (526 nm) under different concentrations of Eu³⁺.

FIG. 13 is a graph of the colorimetric response for different metals. The concentration of ligand and Au³⁺ was 400 μM. The binding curves have been offset for clarity.

FIG. 14 shows a colorimetric assay with tunable dynamic range. The response curve of the colorimetric assay under different concentrations of Au³⁺ and 3,4,3-LI(1,2-HOPO).

### DETAILED DESCRIPTION

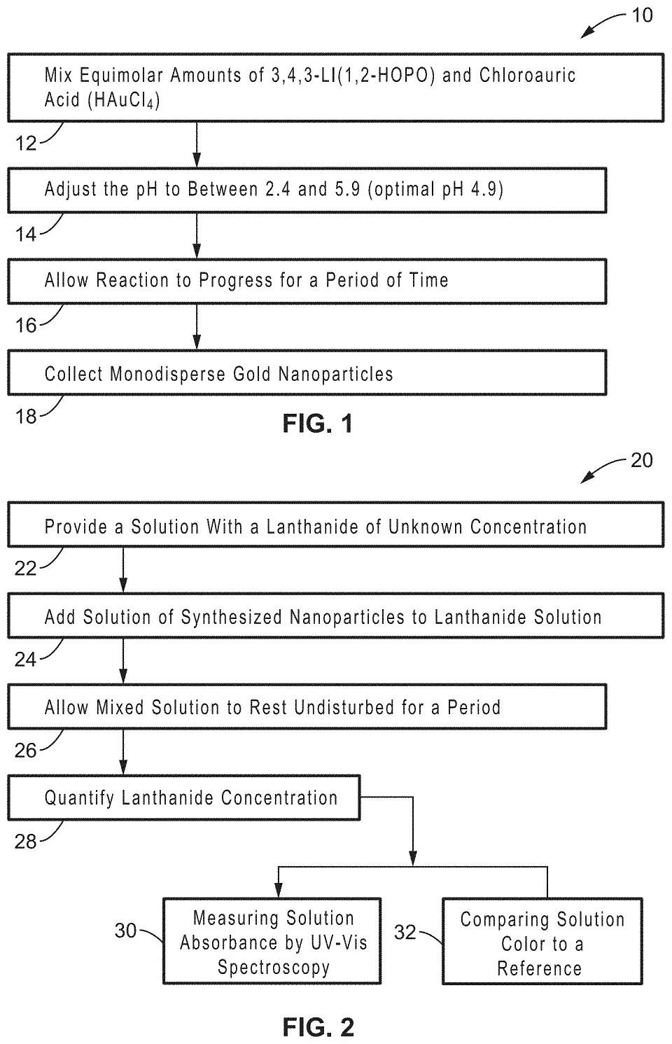

Referring more specifically to the drawings, for illustrative purposes, systems and methods for lanthanide and actinide element detection and quantification are generally shown. Several embodiments of the technology are described generally in FIG. 1 to FIG. 14 to illustrate the characteristics and functionality of the devices, systems and methods. It will be appreciated that the methods may vary as to the specific steps and sequence and the systems and apparatus may vary as to structural details without departing from the basic concepts as disclosed herein. The method steps are merely exemplary of the order that these steps may occur. The steps may occur in any order that is desired, such that it still performs the goals of the claimed technology.

Turning now to FIG. 1, an embodiment of the method 10 for the fabrication of functionalized metal nanoparticles is shown schematically. The methods are illustrated with the synthesis of functionalized gold nanoparticles.

At block 12, equimolar amounts of 3,4,3-LI(1,2-HOPO) and chloroauric acid (HAuCl₄) are obtained. The materials are mixed and the solution is placed into a suitable container. The pH of the mixture is then adjusted to between approximately pH 2.4 and pH 5.9 at block 14. A pH of 4.9 has been found to be optimal for the final solution formed at block 14. Hydrogen chloride is one preferred acid that can be used to adjust the pH. However, other acids can be used. Sodium hydroxide can also be used to adjust the pH to within the desired range.

In one embodiment, hydrogen chloride or sodium hydroxide is added to the solution of hydroxypyridinone ligands (e.g., 3,4,3-LI(1,2-HOPO)) to adjust the pH prior to adding the HAuCl₄ and the pH of the solution is adjusted to a range of about pH 2.4 to pH 5.9, with pH 4.9 particularly preferred. In another embodiment, the mixture of hydroxypyridinone ligands with chloroauric acid (HAuCl₄) is not pH adjusted.

In another embodiment, the HAuCl₄ and hydroxypyridinone components are mixed for a period of about 5 seconds to about 15 seconds; with mixing about 10 seconds preferred. However, mixing times can vary depending on reaction conditions.

The final solution is left to progress and incubate for a period of time, typically for about 45 minutes to one hour or less at block 16. However, incubation times can vary depending on reaction conditions. The final solution is preferably allowed to rest undisturbed to produce the final nanoparticles at block 16 of FIG. 1. The 3,4,3-LI(1,2-HOPO) acts as both a reducing and a stabilizing agent during the growth of the nanoparticles.

At block 18, the monodisperse functionalized gold nanoparticles are collected for subsequent lanthanide and actinide sensing. The collected nanoparticles with hydroxypyridinone ligands disposed on the surface can be washed and redispersed into a solution and stored to be used later for sensing.

The functionalized gold metal nanoparticles produced by the methods have a size that is preferably in the range of about 15 nanometers to about 30 nanometers in diameter. A diameter of about 26 nanometers is particularly preferred. While particles of sized within this range are typical, larger particles (up to 100 nanometers) and smaller particles (down to 2 nanometers) may also be produced.

Although the embodiment of the method 10 described in FIG. 1 produces a composition of matter for sensing of a gold nanoparticle with 3,4,3-LI(1,2-HOPO) ligands disposed on the surface, it will be understood that the methods can be adapted to produce nanoparticles other than gold and hydroxypyridinone ligands other than 3,4,3-LI(1,2-HOPO) including other hydroxypyridinone ligands such as TREN-(Me-3,2-HOPO) and 5-LIO(Me-3,2-HOPO).

Dispersions of the functionalized nanoparticles can be stored and used with lanthanide and actinide sensing of collected samples. Suitable kits with functionalized nanoparticles for detecting and quantifying the f-block metal ions also form part of the present technology. Kits should contain instructions, appropriate reagents, and one or more of the presently disclosed functionalized gold nanoparticles in an appropriate storage form. Other kits may include the components for synthesizing functionalized nanoparticles as described herein. Such kits can be prepared from readily available materials and reagents and can come in a variety of embodiments. The contents of the kit will depend on the design of the assay protocol or reagents for detection or measurement.

A sensing protocol 20 using the synthesized nanoparticles for detecting and quantifying lanthanides and actinides is shown schematically in FIG. 2. A sample of that may contain a lanthanide or actinide of unknown concentration for evaluation is provided at block 22. Sample solutions provided at block 22 may come from a wide variety of sources such as wastewater, sea water, urine, soil, saliva, cells, food, beverages and pharmaceuticals.

At block 24, the synthesized nanoparticles are added into the sample containing the unknown presence or concentration of lanthanide or actinide. The mixed solution of nanoparticles and sample is then preferably left undisturbed for a period of time at block 26 to allow reactions or interactions with the functionalized nanoparticle sensor to take place. Normally, to produce a detectable response, reaction times are about 10 minutes or less and depend on the sensing conditions.

The lanthanide or actinide concentration can be detected and quantified at block 28. Generally, the higher the concentration of lanthanides, the stronger the nanoparticle color change becomes (red to blue), for example.

If the functionalized gold nanoparticles are used to precisely quantify the lanthanide concentration, the solution absorbance is measured by UV-Vis spectroscopy at block 30. In this case, the nanoparticle absorbance changes proportionally to the lanthanide concentration.

If the functionalized gold nanoparticles are used to quantify the lanthanide concentration by observation (i.e., without an instrument), the solution color is compared to references of known concentrations at block 32. Here, the nanoparticle solution color changes from red to blue as the concentration of lanthanide increases. References that are used for comparison, are generally optically graded scales representing f-block elements of various known concentrations.

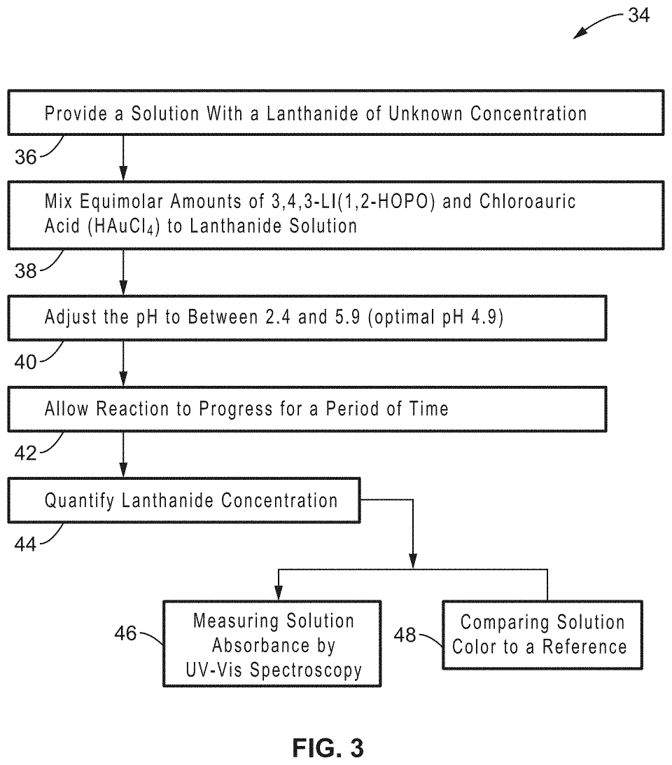

A second sensing protocol 34 for lanthanide and actinide sensing is shown schematically in FIG. 3. Generally, the nanoparticles are grown in situ in the sample solution. Because the lanthanides or actinides quench the growth of gold nanoparticles, the higher the lanthanide or actinide concentration, the less intense the nanoparticle color becomes (e.g. from strong red to colorless).

In this embodiment, a sample solution containing an unknown concentration of f-block elements is provided at block 36. Then, equimolar amounts of hydroxypyridinone (i.e., 3,4,3-LI(1,2-HOPO) and chloroauric acid (HAuCl₄) are added into the sample containing the unknown concentration of f-block element at block 38.

At block 40 hydrogen chloride or other suitable acid is added to the solution to adjust the pH between 2.4 and 5.9 with an optimal pH of about pH 4.9. At block 44, the reaction is left to progress at room temperature for period of time from several minutes to about 45 minutes to an hour to allow sensor particle formation. If the functionalized gold nanoparticles are to be used to precisely quantify the lanthanide or actinide concentration, the solution absorbance is measured by UV-Vis spectroscopy at block 46. If the gold nanoparticles are used to quantify the lanthanide concentration by visual evaluation (i.e., without an instrument), the solution color is compared to a set of reference colors at block 48.

It can be seen that the one-pot synthesis of gold nanoparticles functionalized with hydroxypyridinone chelators provides a simple lanthanide and actinide sensing complex and sensing schemes. Functionalized nanoparticles permit the detection and quantification of f-block elements in a sample with a simple application of nanoparticles and a visual or spectral analysis of the mixture. Since the protocol allows for the straightforward synthesis of AuNPs that feature a chelator with high affinity for f-block elements on the surface, new opportunities in biosensing and therapeutics are presented.

The methods can also combine the one-pot synthesis and functionalization of AuNPs with a high performing chelator tuned specifically for f-block elements with spectroscopy detection or a simple comparative visual quantification in one sequence of steps. The inhibition of particle growth by lanthanides or actinides is used as the colorimetric sensing principle, and the dynamic range of the assay can be tuned by varying the concentrations of Au³⁺ and hydroxypyridinone chelators like 3,4,3-LI(1,2-HOPO), reaching the limits of detection (3 μM) necessary to quantify lanthanides in waste from industrial processes, for example.

The technology described herein may be better understood with reference to the accompanying examples, which are intended for purposes of illustration only and should not be construed as in any sense limiting the scope of the technology described herein as defined in the claims appended hereto.

### Example 1

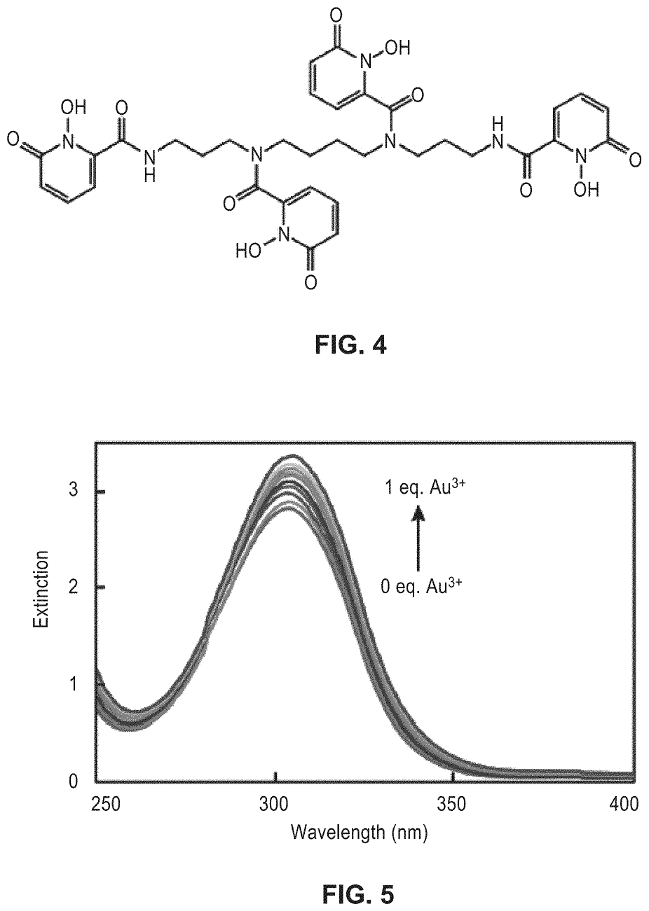

In order to demonstrate the functionality of the detection methods, the solution affinity of Au³⁺ and the therapeutic chelating agent 3,4,3-LI(1,2-HOPO) was demonstrated. The chemical structure of the chelator is shown in FIG. 4.

Complexation with Au³⁺ was observed even at acidic pH (2.2), where most of the HOPO binding units were protonated. Therefore, only a small fraction of partially deprotonated 3,4,3-LI(1,2-HOPO) was necessary to trigger the complexation, indicating a high stability between the cation and the chelator. Above pH 2.4, complexation was followed by the reduction of Au³⁺ to Au⁰, and subsequent formation of AuNPs. During the growth of the particles, 3,4,3-LI(1,2-HOPO) was observed to act as both reducing and stabilizing agent.

The interaction between Au³⁺ and 3,4,3-LI(1,2-HOPO) was initially studied at pH 2.2 because the chelator has shown different complexation behaviors (from total binding to no interaction) with several metals at this acidic pH. It has also been shown that the 1,2-HOPO chromophores have an extinction band at 303 nm, which is sensitive to metal binding, thus the complexation of Au³⁺ was studied by spectrophotometric titration from 250 nm to 400 nm as seen in FIG. 5.

It was observed that the addition of one equivalent of Au³⁺ to a solution of 3,4,3-LI(1,2-HOPO) increased the intensity of the chelator band by 10±2%, indicating formation of the complex. Two isosbestic points at 281 nm and 327 nm were observed in the results of FIG. 5, which suggest that only two species, such as 3,4,3-LI(1,2-HOPO) and Au³⁺, formed the complex.

The proton independent stability constants (i.e., equilibrium constant for the formation of a complex) between Au³⁺ and the chelator were determined via refinement of spectrophotometric titration data to be log β₁₁₀=16.03±0.04 for the [Au-3,4,3-LI(1,2-HOPO)]⁻ complex and log β₁₁₁=17.98±0.04 for the [Au-3,4,3-LI(1,2-HOPO)H] complex. The log β₁₁₀ value was shown to be smaller than corresponding stability constants reported for lanthanides (ranging from 16.4 to 22.2), which are harder trivalent cations (weakly polarizable) that form more stable complexes with the HOPO chelator.

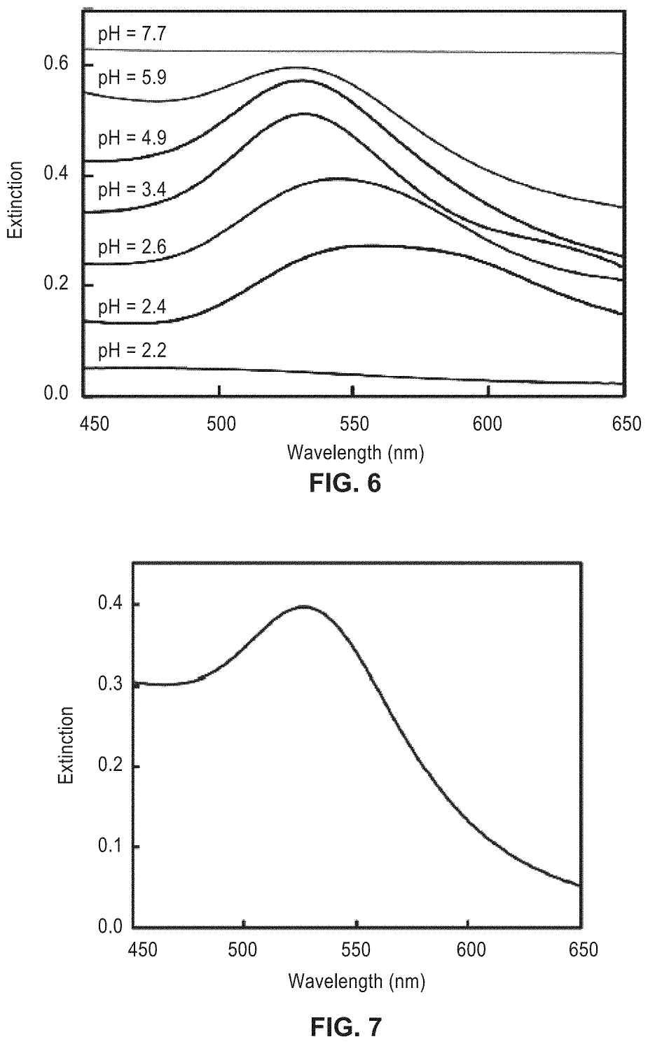

Since 3,4,3-LI(1,2-HOPO) binding affinities to cations are sensitive to the protonation of the 1,2-HOPO subunits (pKₐ values ranging from 3.9 to 6.6), the interaction between the ligand and Au³⁺ was further studied at varying pH values. UV-Vis spectra of 1:1 mixtures of Au³⁺ and 3,4,3-LI(1,2-HOPO) at different pH values are displayed in FIG. 6 and FIG. 7. The UV-vis spectrum of AuNPs synthesized at pH 4.9 indicate that they are primarily monodisperse in solution is shown in FIG. 7.

Au³⁺ can be reduced to Au⁰ at acidic pH (from 2.0 to 6.0) by nitrogen-rich molecules, and as a result the UV-Vis spectra of the samples were measured from 450 nm to 700 nm, where colloidal metal Au absorbs. The pH of the samples was adjusted prior to the addition of Au³⁺ with hydrogen chloride or sodium hydroxide, and the cation and chelator were left to react for 1 h. At pH 2.2, no extinction band between 450 nm and 700 nm was observed because the Au³⁺ was complexed by 3,4,3-LI(1,2-HOPO), as described in the previous paragraph, and no reduction occurred. As the pH was systematically increased up to 5.9, an extinction band between 526 nm and 560 nm appeared, which correlates to the localized surface plasmon (LSP) resonances of AuNPs. The LSP band was broad at low pH (from 2.4 to 3.4), indicating particle aggregation and/or polydispersity. As the pH increased up to 4.9, the LSP band centered at 526 nm and became sharper, which suggested the formation of monodisperse and well-dispersed AuNPs. At higher pH values, larger fractions of HOPO moieties are deprotonated, thereby providing stronger electrostatic repulsion between particles that favors AuNP dispersion.

The system was further characterized by cyclic voltammetry under AuNP growth conditions (pH 5.9). Because nanoparticles start forming and depositing on the electrode at this pH, affecting the measurements, the solutions were mixed and left to stabilize for only 5 minutes before performing the voltammetry. A positive half-wave potential (i.e. difference between cathodic and anodic peaks, E_{1/2}), of 0.685 V was measured, indicating that the reaction is thermodynamically favorable. The growth of AuNPs within this range of pH (from 2.4 to 5.9) is consistent with the synthesis of particles made with other reducing agents. Further increase of pH diminished the intensity of LSP band until total disappearance at pH 7.7. The lack of AuNP growth at higher pH is not caused by the formation of Au hydroxides, since the speciation diagram calculated with the stability constants show that Au³⁺ and Au³⁺-3,4,3-LI(1,2-HOPO) complexes are the primary species in solution between pH 1 and pH 10.

### Example 2

To demonstrate the ability to grow metal nanoparticles with 3,4,3-LI(1,2-HOPO), AuNPs were synthesized by adding 1 equivalent of HAuCl₄ to 3,4,3-LI(1,2-HOPO) solutions (400 μM final concentration). The pH of the solutions was adjusted with hydrogen chloride or sodium hydroxide prior the addition of Au³⁺. The resulting solutions were mixed for 10 s and left undisturbed at room temperature for 1 h. 100 μL of the AuNP solutions were used to record UV-Vis spectra with a SpectraMax iD3 Multi-Mode Microplate Reader. Transmission electron microscopy (TEM) was performed using a FEI Them IS (Thermo Fisher Scientific, Waltham, Mass.) with an image aberration corrector, operated at 300 kV. AuNP morphology and elemental composition were characterized by high-angle annular dark field (HAADF) and energy dispersive spectroscopy (EDS) in STEM mode. High-resolution TEM (HR-TEM) micrographs were acquired on a FEI Ceta camera. Hydrodynamic diameter and zeta potential values were measured with a ZetaPlus BI-90 Plus (Brookhaven Instruments Corporation, Holtsville, N.Y.).

To quantify the yield of the AuNP synthesis, the particles were washed by centrifugation (12000×g) for 15 min to remove unreacted Au³⁺ and re-dispersed in Milli-Q water. Then 50 μL of washed AuNPs were digested in 100 μL of nitric acid (70%) and 100 μL of hydrogen chloride (6 N) for 30 min at room temperature and re-dispersed in 4.75 mL of Milli-Q water. The final concentration of Au in solution was quantified by Inductively Coupled Plasma Optical Emission Spectroscopy (ICP-OES, Perkin Elmer, Waltham, Mass.).

Cyclic voltammetry (CV) data were collected at room temperature in water using 1 M of CaCl₂) as a supporting electrolyte with sweep rates of 50 mV/s over the range −200 to 1000 mV on a Pine Research (Durham, N.C.) WaveNow potentiostat. Gold ceramic screen-printed electrodes were used for CV measurements with an Ag/AgCl reference electrode and the electrode surfaces were checked in between each measurement for potential deactivation using a 3 mM Fe(CN)₆⁴⁻/Fe(CN)₆³⁻ solution. CVs of blank solutions were also measured to subtract the influence of non-faradic current, thus all spectra presented herein are background subtracted.

For characterization of the produced nanoparticles, AuNPs synthesized at pH 4.9 were selected because their UV-vis spectrum indicated they were primarily monodispersed in solution as shown in FIG. 6. Scanning transmission electron microscope (STEM) images in dark field mode showed quasi-spherical AuNPs with average diameters of 24±4 nm and a small fraction with triangular shapes. Both size dispersion (16.5%, calculated as percent of standard deviation) and crystal structure (the particles were polycrystalline with multiply twinned structures, appeared to be very similar to the ones obtained via the Turkevich method by citrate reduction of gold salts.

Spectral analysis and mapping through the energy-dispersive X-ray spectroscopy (EDS) mode of the STEM confirmed that the produced nanoparticles were made of Au. The yield of the particle synthesis defined as the percentage of Au³⁺ that was reduced to metal and formed Au nanoparticles was 87.2%.

A zeta potential value of −22.9±2.6 mV was recorded after washing the particles and resuspending them in Milli-Q water. The negative zeta potential of the particles confirmed their stability in solution, which was corroborated by the invariability of the LSP band after one month, suggesting that the chelator acted as stabilizing agent.

The presence of the 3,4,3-LI(1,2-HOPO) on the metal surface was confirmed by fluorescence spectroscopy, exploiting the chelator sensitization properties. The complex that is formed between 3,4,3-LI(1,2-HOPO) and Eu³⁺ has a characteristic emission band centered at 618 nm, which was recorded after the addition of Eu³⁺ to the washed particle solution. The addition of Eu³⁺ also caused the aggregation of the particles, as observed in their UV-vis spectrum. Although AuNPs can quench the emission of fluorophores located on their surface, fluorescence enhancement occurs when the particles are aggregated because of the formation of hotspots. Hence, the chelator acted as both reducing and stabilizing agent in the growth of the particles.

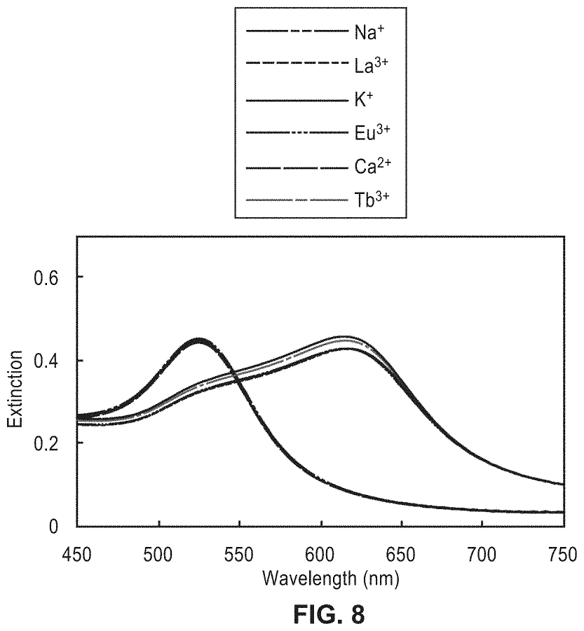

The dual role of 3,4,3-LI(1,2-HOPO) is also consistent with the synthesis of AuNPs with other chelates such as EDTA. An AuNP aggregation assay confirmed that the 3,4,3-LI(1,2-HOPO) on the Au particle surface preserved selectivity toward f-block elements over lighter ones as only lanthanides induced nanoparticle aggregation. The preservation of selectivity is demonstrated in the UV-Vis spectra of FIG. 8.

The growth kinetics of the Au nanoparticles using the method were also evaluated. A 1 mL solution containing 400 μM of HAuCl₄ and 3,4,3-LI(1,2-HOPO) at pH 4.9 was prepared at room temperature and immediately transferred to a plastic cuvette. Solution state UV-Vis spectra (from 450 nm to 650 nm) were recorded with a Cary 4000 UV-vis-NIR spectrophotometer (Agilent, Santa Clara, Calif.) at fixed intervals (one spectrum every min).

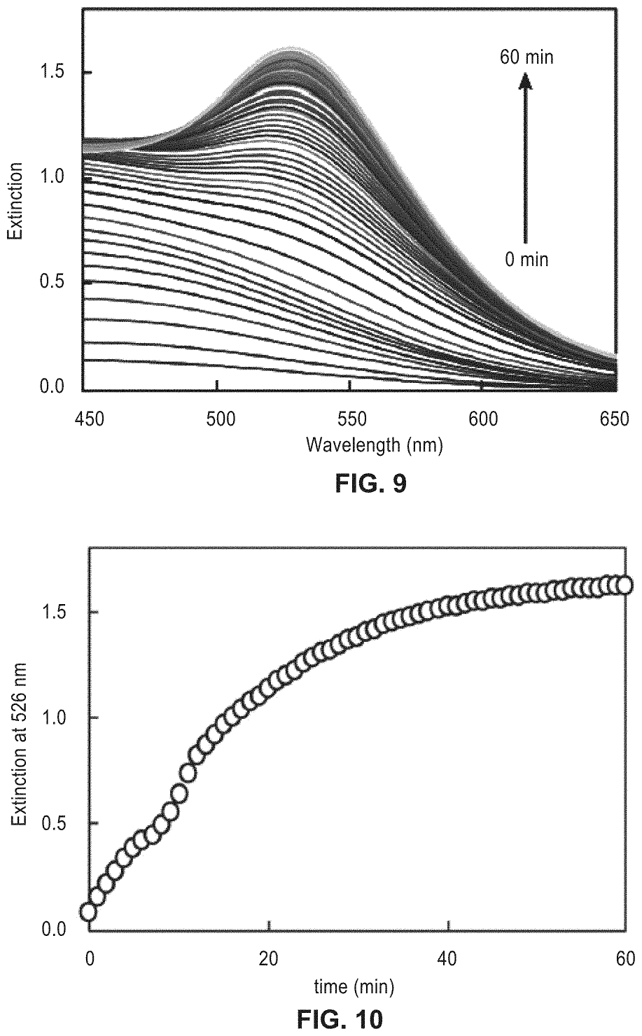

To further understand the growth mechanism of the particles, the growth kinetics were tested by UV-vis spectroscopy as seen in FIG. 9 and FIG. 10. An extinction band below 500 nm, which is characteristic of Au⁰ interband transitions, appeared in the first minute after the addition of Au³⁺ to the growth solution, resulting in a faint red color visible by the naked eye within 5 minutes, and the extinction intensity of this band increased as the reaction progressed. After 18 minutes, a LSP band at 518 nm arose, which denoted the formation and growth of colloidal Au above 2 nm (size necessary to hold LSP resonances). As the reaction continued, the LSP increased in intensity and shifted to 526 nm, suggesting particle growth. The reaction ended after 50 minutes when the position and intensity of the LSP band had stabilized.

To evaluate the interaction between AuNPs and cations, AuNPs were washed by centrifugation (12000×g) for 15 minutes to remove unreacted Au³⁺, and re-dispersed in MES buffer (0.1 M, pH 6.0). 1 μL of cation solution was added to 100 μL of the AuNP solution, mixed, and incubated at room temperature for 10 minutes. The final concentration of cations was either 10 μM or 100 μM. The fluorescence and UV-Vis spectra of solutions were recorded with a SpectraMax iD3 Multi-Mode Microplate Reader.

Accordingly, it was demonstrated that stable metal nanoparticles functionalized with the hydroxypyridinone (3,4,3-LI(1,2-HOPO) can be produced with a simple process and used as a lanthanide sensor.

### Example 3

To demonstrate the single pot synthesis and detection with nanoparticles that are grown in situ in the sample solution, competition experiments were performed to illustrate that chelation of the Au³⁺ is necessary for the synthesis of Au nanoparticles. Because the lanthanides quench the growth of gold nanoparticles, the higher the lanthanide concentration, the less intense the nanoparticle color becomes (from strong red to colorless) and this process can be used to quantify an analyte concentration.

To generally assess the effects of changing input parameters and the presence of competing cations, solutions containing divalent (Ca²⁺ or Cu²⁺) or trivalent (La³⁺, Eu³⁺, Tb³⁺ or Gd³⁺) cations were spiked into a set of 3,4,3-LI(1,2-HOPO) solutions at pH 4.9 and left to react for 10 min. Fixed amounts of HAuCl₄ were added into the mixtures for a final volume of 100 μL where ligand and Au³⁺ concentrations were 400 μM, and the cation concentrations ranged from 0 to 1.25 equivalents. The solutions were mixed after the addition of Au³⁺ and incubated at room temperature for 1 hour before recording UV-Vis spectra from 450 nm to 650 nm with a SpectraMax iD3 Multi-Mode Microplate Reader. The measurement variation is reported as error bars in the figures, which represent one standard deviation of the measurements. For the tunable dynamic range assessment, the same protocol was followed varying the amount of both 3,4,3-LI(1,2-HOPO) and HAuCl₄ in the growth solutions.

Since the interaction between Au³⁺ and 3,4,3-LI(1,2-HOPO) resulted in both complex formation and particle growth, it is important to show that the chelation of the Au³⁺ was necessary to synthesize AuNPs rather than both events being independent.

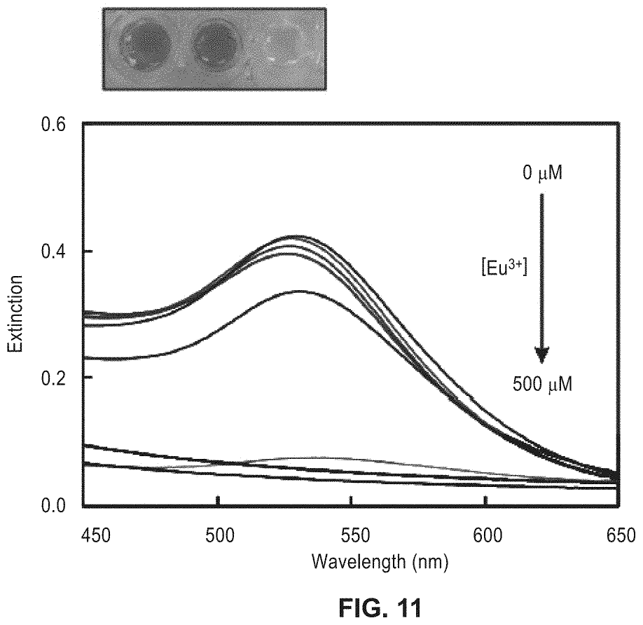

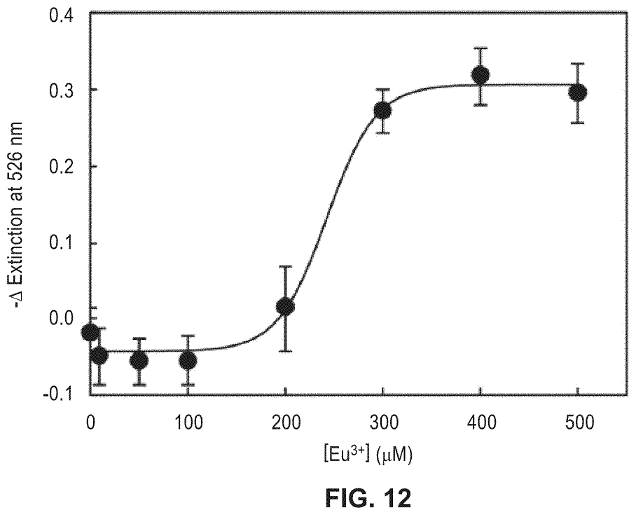

Competition experiments were performed with Eu³⁺ that has a thermodynamic stability constant with 3,4,3-LI(1,2-HOPO) that is higher (log β₁₁₀ of 20.2) than the one that was determined for Au³⁺. It was previously shown that the complexation of Eu³⁺ by 3,4,3-LI(1,2-HOPO) occurs within 10 min, thus Eu³⁺ was added to the solution and allowed to interact with the chelate for 10 min prior the addition of Au³⁺. After the Au³⁺ addition, the mixture was left to react for 1 h and then characterized by UV-Vis spectroscopy. FIG. 11 shows that increasing the concentration of Eu³⁺ prevented the formation AuNPs as the LSP band decreased in intensity and red-shifted. This confirmed that the reduction of Au³⁺ to Au⁰ by 3,4,3-LI(1,2-HOPO) required the complexation of the Au³⁺, since the presence of a cation with higher affinity for the chelator hindered the particle growth. The variations in the LSP band due to the presence of Eu³⁺ during AuNP formation were examined as a colorimetric sensing principle, where the color change of the solution could be used to quantify the analyte concentration as shown in FIG. 12.

Although lanthanides are usually quantified by ICP-MS and ICP-OES, these instruments are intensive in cost and personnel. Thus, a rapid and straightforward assay capable of quantifying lanthanide concentrations with minimal instrumentation or simply by visual analysis would be highly beneficial. Conventional colorimetric assays based on AuNP aggregation only change in color as the concentration of analyte increases. However, as shown in FIG. 11 and FIG. 12, there is a simultaneous color change (shift of the LSP band) and color disappearance (decrease of LSP intensity), which provide a more distinguishable analytical response, particularly when used as a point-of-care assay read by visual observation.

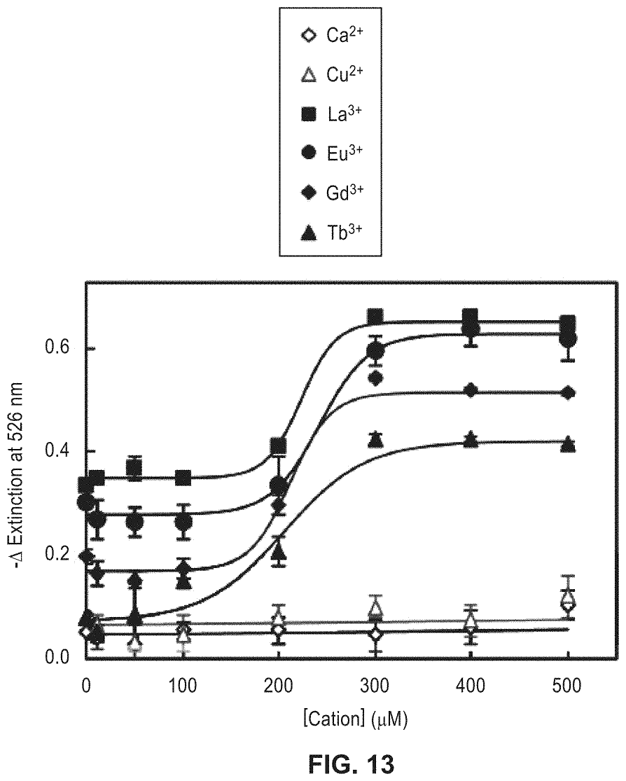

The quantification accuracy with the protocol was 87%, which was estimated by analyzing a reference sample (250 μM Eu³⁺), and it was similar to other nanoparticle-based colorimetric assay accuracies. As a result, we explored the quenching of AuNP growth as a sensing principle with other lanthanides, such as La³⁺, Gd³⁺ and Tb³⁺, which also have higher thermodynamic stability constants with 3,4,3-LI(1,2-HOPO) than Au³⁺. As controls Ca²⁺ and Cu²⁺, two divalent cations commonly found in buffers, were used that are softer than Au³⁺ and were expected to have a lower affinity for the chelator. The binding curves showed similar responses for all lanthanides and are depicted in FIG. 13. Ca²⁺ and Cu²⁺ did not induce any significant changes as they did not prevent the formation of AuNPs.

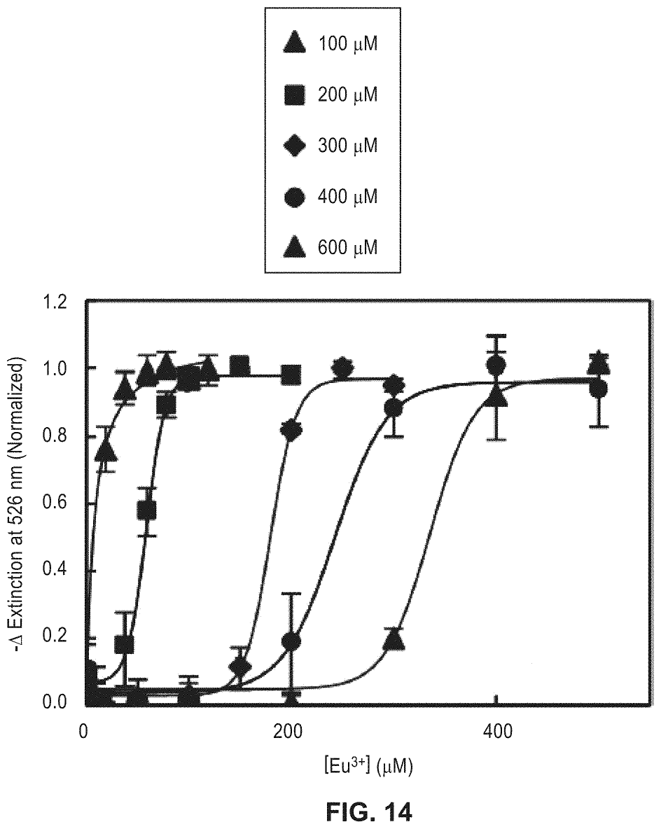

The U.S. Department of Energy (DOE) has highlighted the lanthanides as critical materials for clean energy technologies with high risk of supply disruptions in the near future. Hence, new strategies to quantify and recycle rare-earth elements in waste streams are required. One major challenge when characterizing lanthanides in solution is their wide concentration variations depending on the sample (i.e., from low μM to high μM or mM range in high level waste waters from nuclear reactors). To take full advantage of a colorimetric assay, tunable limits of detection and dynamic ranges are necessary. Towards this goal, both the limit of detection and the dynamic range were adjusted by changing the concentration of chelate and Au³⁺ to demonstrate the adaptability of the process. FIG. 14 depicts the colorimetric response at five different concentrations (from 100 to 600 μM HAuCl₄ and 3,4,3-LI(1,2-HOPO)). The limit of detection shifted from 3 μM to 302 μM as the concentrations of Au³⁺ and chelator increased. Thus, the combination of the five solutions yielded a colorimetric assay capable of covering the content of several lanthanides in waste from industrial processes (e.g., 3.1 μM Tb³⁺, 105 μM Gd³⁺ and 148 μM Eu³⁺).

Accordingly, the inhibition of particle growth by lanthanides can be used as a colorimetric sensing principle, and the dynamic range of the assay can be tuned by varying the concentrations of Au³⁺ and 3,4,3-LI(1,2-HOPO) thereby reaching the limits of detection (3 μM) that are necessary to quantify typical lanthanide concentrations in waste from industrial processes.

From the description herein, it will be appreciated that the present disclosure encompasses multiple embodiments of the technology which include, but are not limited to, the following:

A lanthanide or actinide sensing construct, comprising: (a) a nanoparticle of gold metal; and (b) a plurality of hydroxypyridinone ligands disposed on a surface of the gold metal nanoparticle; wherein an interaction of a lanthanide or actinide with the construct in a solution can be optically detected.

The construct of any preceding or following embodiment, wherein the gold metal nanoparticles have a diameter in the range of about 15 nanometers to about 30 nanometers in diameter.

The construct of any preceding or following embodiment, wherein the gold metal nanoparticles have a diameter of about 26 nanometers.

The construct of any preceding or following embodiment, wherein the hydroxypyridinone ligands comprise octadentate hydroxypyridinone ligands.

The construct of any preceding or following embodiment, wherein the hydroxypyridinone ligands are selected from the group of ligands consisting of 3,4,3-LI(1,2-HOPO), TREN-(Me-3,2-HOPO), and 5-LIO(Me-3,2-HOPO).

A method for fabricating a lanthanide or actinide sensing construct, the method comprising: (a) mixing a solution of hydroxypyridinone ligands with chloroauric acid (HAuCl₄); (b) reacting the mixture for one hour or less; and (c) collecting gold metal nanoparticles with a plurality of hydroxypyridinone ligands disposed on a surface of each nanoparticle.

The method of any preceding or following embodiment, wherein the hydroxypyridinone ligands comprise octadentate hydroxypyridinone ligands.

The method of any preceding or following embodiment, wherein the hydroxypyridinone ligands are selected from the group of ligands consisting of 3,4,3-LI(1,2-HOPO), TREN-(Me-3,2-HOPO), and 5-LIO(Me-3,2-HOPO).

The method of any preceding or following embodiment, wherein the HAuCl₄ and the solution of 3,4,3-LI(1,2-HOPO) are in equimolar amounts.

The method of any preceding or following embodiment, further comprising adding hydrogen chloride or sodium hydroxide to the solution of 3,4,3-LI(1,2-HOPO) to adjust the pH prior to adding the HAuCl₄; wherein the pH of the solution is adjusted to a range of about pH 2.4 to pH 5.9.

The method of any preceding or following embodiment, wherein the pH of the solution is adjusted to about pH 4.9.

The method of any preceding or following embodiment, further comprising adding hydrogen chloride or sodium hydroxide to the solution of 3,4,3-LI(1,2-HOPO) to adjust the pH prior to adding the HAuCl₄.

The method of any preceding or following embodiment, further comprising washing the gold nanoparticles in water; and re-dispersing the washed nanoparticles in water to form a nanoparticle dispersion.

The method of any preceding or following embodiment, further comprising mixing HAuCl₄ and hydroxypyridinone for about 5 seconds to about 15 seconds; and leaving the mixture undisturbed for 60 minutes or less before collecting the nanoparticles.

A method of detecting and quantifying the presence of a lanthanide or actinide in a sample, the method comprising: (a) synthesizing functionalized gold metal nanoparticles with a plurality of hydroxypyridinone ligands disposed on a surface of the gold metal nanoparticle; (b) adding the functionalized gold metal nanoparticles to a sample solution with a lanthanide or actinide of unknown concentration to produce a mixture; (c) incubating the mixture for 10 minutes or less; and (d) quantifying the lanthanide concentration of the incubated mixture.

The method of any preceding or following embodiment, wherein the lanthanide concentration of the incubated mixture is quantified by absorbance measurements by UV-Vis spectroscopy.

The method of any preceding or following embodiment, wherein the lanthanide concentration of the incubated mixture is quantified by visual comparison with a reference.

The method of any preceding or following embodiment, wherein the hydroxypyridinone ligands are selected from the group of ligands consisting of 3,4,3-LI(1,2-HOPO), TREN-(Me-3,2-HOPO), and 5-LIO(Me-3,2-HOPO).

The method of any preceding or following embodiment, wherein the gold metal nanoparticles have a diameter in the range of about 15 nanometers to about 30 nanometers in diameter.

A method of detecting and quantifying the presence of a lanthanide or actinide in a sample, the method comprising: (a) providing a sample solution with a lanthanide or actinide of unknown concentration; (b) adding equal molar amounts of hydroxypyridinone ligands and chloroauric acid (HAuCl₄) to the sample solution to form a mixture; (c) adjusting the pH of the mixture to within a range of about pH 2.4 to about pH 5.9 to produce a pH adjusted mixture; (d) incubating the pH adjusted mixture undisturbed for 60 minutes or less; and (e) quantifying the lanthanide or actinide concentration of the incubated mixture.

The method of any preceding or following embodiment, further comprising mixing the pH adjusted mixture for about 5 seconds to about 15 seconds prior to incubation.

The method of any preceding or following embodiment, wherein the pH of the solution is adjusted to about pH 4.9.

The method of any preceding or following embodiment, wherein the hydroxypyridinone ligands comprise octadentate hydroxypyridinone ligands.

The method of any preceding or following embodiment, wherein the hydroxypyridinone ligands are selected from the group of ligands consisting of 3,4,3-LI(1,2-HOPO), TREN-(Me-3,2-HOPO), and 5-LIO(Me-3,2-HOPO).

The method of any preceding or following embodiment, wherein the lanthanide or actinide concentration of the incubated mixture is quantified by absorbance measurements by UV-Vis spectroscopy.

The method of any preceding or following embodiment, wherein the lanthanide or actinide concentration of the incubated mixture is quantified by visual comparison with a reference.

As used herein, term “embodiment” is intended to include, without limitation, embodiments, implementations, examples, or other forms of practicing the technology described herein.

As used herein, the singular terms “a,” “an,” and “the” may include plural referents unless the context clearly dictates otherwise. Reference to an object in the singular is not intended to mean “one and only one” unless explicitly so stated, but rather “one or more.”

Phrasing constructs, such as “A, B and/or C”, within the present disclosure describe where either A, B, or C can be present, or any combination of items A, B and C. Phrasing constructs indicating, such as “at least one of” followed by listing a group of elements, indicates that at least one of these group elements is present, which includes any possible combination of the listed elements as applicable.

References in this disclosure referring to “an embodiment”, “at least one embodiment” or similar embodiment wording indicates that a particular feature, structure, or characteristic described in connection with a described embodiment is included in at least one embodiment of the present disclosure. Thus, these various embodiment phrases are not necessarily all referring to the same embodiment, or to a specific embodiment which differs from all the other embodiments being described. The embodiment phrasing should be construed to mean that the particular features, structures, or characteristics of a given embodiment may be combined in any suitable manner in one or more embodiments of the disclosed apparatus, system or method.

As used herein, the term “set” refers to a collection of one or more objects. Thus, for example, a set of objects can include a single object or multiple objects.

Relational terms such as first and second, top and bottom, and the like may be used solely to distinguish one entity or action from another entity or action without necessarily requiring or implying any actual such relationship or order between such entities or actions.

The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element.

As used herein, the terms “approximately”, “approximate”, “substantially”, “essentially”, and “about”, or any other version thereof, are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, “substantially” aligned can refer to a range of angular variation of less than or equal to ±10°, such as less than or equal to ±5°, less than or equal to ±4°, less than or equal to ±3°, less than or equal to ±2°, less than or equal to ±1°, less than or equal to ±0.5°, less than or equal to ±0.1°, or less than or equal to ±0.05°.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

The term “coupled” as used herein is defined as connected, although not necessarily directly and not necessarily mechanically. A device or structure that is “configured” in a certain way is configured in at least that way, but may also be configured in ways that are not listed.

Benefits, advantages, solutions to problems, and any element(s) that may cause any benefit, advantage, or solution to occur or become more pronounced are not to be construed as a critical, required, or essential features or elements of the technology describes herein or any or all the claims.

In addition, in the foregoing disclosure various features may grouped together in various embodiments for the purpose of streamlining the disclosure. This method of disclosure is not to be interpreted as reflecting an intention that the claimed embodiments require more features than are expressly recited in each claim. Inventive subject matter can lie in less than all features of a single disclosed embodiment.

The abstract of the disclosure is provided to allow the reader to quickly ascertain the nature of the technical disclosure. It is submitted with the understanding that it will not be used to interpret or limit the scope or meaning of the claims.

It will be appreciated that the practice of some jurisdictions may require deletion of one or more portions of the disclosure after that application is filed. Accordingly, the reader should consult the application as filed for the original content of the disclosure. Any deletion of content of the disclosure should not be construed as a disclaimer, forfeiture or dedication to the public of any subject matter of the application as originally filed.

The following claims are hereby incorporated into the disclosure, with each claim standing on its own as a separately claimed subject matter.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

All structural and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for.”

What is claimed is:

1. A lanthanide or actinide sensing construct, comprising:
(a) a nanoparticle of gold metal; and
(b) a plurality of hydroxypyridinone ligands disposed on a surface of the gold metal nanoparticle;
wherein an interaction of a lanthanide or actinide with the construct in a solution can be optically detected.

2. The construct of claim 1, wherein said gold metal nanoparticles have a diameter in the range of about 15 nanometers to about 30 nanometers in diameter,

3. The construct of claim 1, wherein said hydroxypyridinone ligands comprise octadentate hydroxypyridinone ligands.

4. The construct of claim 1, wherein said hydroxypyridinone ligands are selected from the group of ligands consisting of 3,4,3-LI(1,2-HOPO), TREN-(Me-3,2-HOPO), and 5-LIO(Me-3,2-HOPO).

5. A method for fabricating a lanthanide or actinide sensing construct, the method comprising:
(a) mixing a solution of hydroxypyridinone ligands with chloroauric acid (HAuCl₄);
(b) reacting the mixture for one hour or less; and
(c) collecting gold metal nanoparticles with a plurality of hydroxypyridinone ligands disposed on a surface of each nanoparticle.

6. The method of claim 5, wherein said hydroxypyridinone ligands are selected from the group of ligands consisting of 3,4,3-LI(1,2-HOPO), TREN-(Me-3,2-HOPO), and 5-LIO(Me-3,2-HOPO).

7. The method of claim 6, wherein the HAuCl₄ and the solution of 3,4,3-LI(1,2-HOPO) are in equimolar amounts.

8. The method of claim 7, further comprising:
adding hydrogen chloride or sodium hydroxide to the solution of 3,4,3-LI(1,2-HOPO) to adjust the pH prior to adding the HAuCl₄;
wherein the pH of the solution is adjusted to a range of about pH 2.4 to pH 5.9.

9. The method of claim 8, further comprising:
adding hydrogen chloride or sodium hydroxide to the solution of 3,4,3-LI(1,2-HOPO) to adjust the pH prior to adding the HAuCl₄.

10. The method of claim 5, further comprising:
washing said gold nanoparticles in water; and
re-dispersing the washed nanoparticles in water to form a nanoparticle dispersion.

11. The method of claim 5, further comprising:
mixing HAuCl₄ and hydroxypyridinone for about 5 seconds to about 15 seconds; and
leaving the mixture undisturbed for 60 minutes or less before collecting the nanoparticles.

12. A method of detecting and quantifying the presence of a lanthanide or actinide in a sample, the method comprising:
(a) synthesizing functionalized gold metal nanoparticles with a plurality of hydroxypyridinone ligands disposed on a surface of the gold metal nanoparticle;
(b) adding the functionalized gold metal nanoparticles to a sample solution with a lanthanide or actinide of unknown concentration to produce a mixture;
(c) incubating the mixture for 10 minutes or less; and
(d) quantifying the lanthanide concentration of the incubated mixture.

13. The method of claim 12, wherein the lanthanide concentration of the incubated mixture is quantified by absorbance measurements by UV-Vis spectroscopy.

14. The method of claim 12, wherein the lanthanide concentration of the incubated mixture is quantified by visual comparison with a reference.

15. The method of claim 12, wherein said hydroxypyridinone ligands are selected from the group of ligands consisting of 3,4,3-LI(1,2-HOPO), TREN-(Me-3,2-HOPO), and 5-LIO(Me-3,2-HOPO).

16. A method of detecting and quantifying the presence of a lanthanide or actinide in a sample, the method comprising:
(a) providing a sample solution with a lanthanide or actinide of unknown concentration;
(b) adding equal molar amounts of hydroxypyridinone ligands and chloroauric acid (HAuCl₄) to the sample solution to form a mixture;
(c) adjusting the pH of the mixture to within a range of about pH 2.4 to about pH 5.9 to produce a pH adjusted mixture;
(d) incubating the pH adjusted mixture undisturbed for 60 minutes or less; and
(e) quantifying the lanthanide or actinide concentration of the incubated mixture.

17. The method of claim 16, further comprising:
mixing the pH adjusted mixture for about 5 seconds to about 15 seconds prior to incubation.

18. The method of claim 16, wherein said hydroxypyridinone ligands are selected from the group of ligands consisting of 3,4,3-LI(1,2-HOPO), TREN-(Me-3,2-HOPO), and 5-LIO(Me-3,2-HOPO).

19. The method of claim 16, wherein the lanthanide or actinide concentration of the incubated mixture is quantified by absorbance measurements by UV-Vis spectroscopy.

20. The method of claim 16, wherein the lanthanide or actinide concentration of the incubated mixture is quantified by visual comparison with a reference.
